(19) United States
(12) Patent Application Publication
(10) Pub. No.: US 2016/0167996 A1
(43) Pub. Date: Jun. 16, 2016

(54) MICROBIAL CONSORTIUM FOR WASTEWATER TREATMENT

(71) Applicant: Chun Yean Chong, Selangor (MY)

(72) Inventor: Chun Yean Chong, Selangor (MY)

(21) Appl. No.: 14/953,918

(22) Filed: Nov. 30, 2015

(30) Foreign Application Priority Data
Dec. 10, 2014 (MY) PI 2014703726

(51) Int. Cl. C02F 3/34; C12N 1/16; C12N 1/12; C12P 7/64; C12N 1/20

(52) U.S. Cl. CPC C02F 3/341; C12P 7/649; C12N 1/20; C12N 1/12; C12N 1/16

(57) ABSTRACT

A liquid composition containing a microbial consortium for treatment of wastewater or water containing organic waste matter, the microbial consortium having at least one saprophytic yeast, at least one photosynthetic microbe, and at least one diazotrophic microbe, wherein the microbes in the consortium are arranged and adapted to form a symbiotic relationship to one another in the wastewater upon administering to and growing in the wastewater. The liquid composition disclosed herein produces an oleaginous biomass suitable for production of biocrude oil.

### FIELD OF INVENTION

This present invention relates to a microbial mixture for effective treatment of wastewater with the intention to produce biocrude oil. More particularly, the microbial mixture is suitable for removal of organic matters in wastewater as well as production of an oleaginous biomass from wastewater. The microbial mixture is a polyculture capable of forming a microecosystem. It is effective in wastewater treatment and is more resilient to different external shocks such as pH and temperature changes.

### BACKGROUND OF THE INVENTION

Wastewater disposal has always been a major concern. For many companies, treatment of wastewater is an expensive liability and can be environmentally hazardous. If wastewater is discharged directly to the environment without proper treatment, the receiving waters, i.e. rivers or sea, would become polluted. Further, untreated wastewater which contain nutrients would fuel the growth of pathogens, thereby causing widespread of water-related diseases. Besides, algae blooms would lead to algal toxin buildup (e.g. toxic algae blooms) in crustaceans and shellfish, which consequently lead to food allergies in human.

Wastewater may be organic or inorganic in nature or a combination of both. Putrescible organic matters in wastewater would adversely affect the water quality of receiving waters. Also, excess organic matters in the wastewater causes decomposers in receiving waters to flourish and consuming oxygen faster than it can be replaced, creating a hypoxic condition resulting in the death of many aquatic organisms.

The conventional method to remove organic matters from wastewater before discharging to receiving waters is treating the wastewater with intense aeration, that is simply infusing the system with more oxygen to fuel the growth of the aerobic microbes so the rate of decomposition is so many times higher than in the natural waterways. The aerobic decomposition depletes the level of organic and inorganic contaminants in the water as the organic materials are consumed for microbial growth or released into the atmosphere in the form of microbial metabolites such as carbon dioxide, consequently, reducing biochemical oxygen demand (BOD) and chemical oxygen demand (COD). In most conventional aerobic wastewater treatment, the rate of decomposition is determined by the level of aeration which increases the level of dissolved oxygen (DO) and nitrification (later denitrification). As oxygen is consumed, decomposition is stifled. This can, however, be improved through the introduction of facultative anaerobes such as fermentative bacteria from genus Acetobacter, Lactobacillus, and Bifidobacteria, and the introduction of symbiotic fungi with these bacteria such as yeast from the genus Candida and Saccharomyces.

Of particular interest is an U.S. Pat. No. 5,811,289, which describes a process of treating dairy industry effluent. The disclosed process comprises inoculating the effluent with a mixture of bacteria and yeasts, of which, many are facultative anaerobes. In the disclosed patent, both classes of microorganisms are capable of living and growing in symbiosis in the effluent, therefore improving the efficiency of the decomposition of organic waste in the system.

Nevertheless, conventional wastewater treatment is still an expensive process to maintain and is detrimental to the environment since greenhouse gasses are produced whenever there is an anaerobic pocket in the system, which cannot be avoided in all aerobic systems. The conventional biological wastewater treatment is simply a liability to the society regardless of how effective it is.

On the other hand, microbial polyculture invention differs from the conventional wastewater treatment in that, the polyculture invention consumes organic and inorganic nutrients in the wastewater to form an oleaginous biomass suitable for the production of biofuel through Hydrothermal Liquefaction (HTL) while reducing BOD and COD in the wastewater. This microbial technology also contain microbes that consumes carbon monoxide and carbon dioxide therefore greatly reducing or potentially releasing no carbon dioxide into the atmosphere or consuming carbon dioxide from the atmosphere.

Therefore, it is desirable to provide a polymicrobial culture which works symbiotically as an microecosystem for both wastewater treatment and biomass production, wherein greenhouse gas emission is limited and need for water aeration and chemical is minimized. Further, the oleaginous biomass is ideal for the production of biocrude oil via hydrothermal liquefaction.

### SUMMARY OF THE INVENTION

The primary object of the invention is to provide a liquid composition containing a microbial consortium for treatment of wastewater containing organic matters. Aerobic digestion of organic matters in the wastewater by the microbes effectively reduces biochemical oxygen demand (BOD) and chemical oxygen demand (COD) of wastewater.

Another object of the invention is to provide a liquid composition containing a microbial consortium capable of growing in wastewater without the need of supplying additional growth nutrients and aeration. The microbes in the consortium form a self-sustaining microecosystem; their own metabolites support the growth of one another. The microbial consortium is capable of breaking down organic matters into carbon substrates, fixing atmospheric nitrogen as well as carbon, and/or providing dissolved oxygen.

Still, one object of the invention is to offer a liquid composition containing a microbial consortium, for treating wastewater containing organic matters, that is genetically stable and more resilient to various environmental shock. The consortium is able to coop with external changes due to its metabolic diversity.

Yet, another object of the invention is to offer a liquid composition containing a microbial consortium capable of producing oleaginous biomass for biocrude oil production while removing unwanted matters in wastewater. The consortium is a polyculture of algae, diazotroph and fungi, when grown in a consortium, algae polyculture tends to have higher lipid content than monocultures of algae. Further, induced stress is not necessary to increase lipid content in algal polyculture as compared to algal monocultures.

At least one of the preceding objects is met, in whole or in part, by the present invention, in which the embodiment of the present invention describes a liquid composition containing a microbial consortium for treatment of wastewater or water containing organic matter, the microbial consortium comprising at least one saprophytic yeast selected from the genus Saccharomyces, Cryptococcus, Rhodosporidium, and Yarrowia; at least one photosynthetic microbe selected from the genus Nannochloropsis, Chlorella, Tetraselmis, Scenedesmus, Rhodopseudomonas, and Rhodobacter; and at least one diazotrophic microbe selected from the genus Azotobacter and Azospirillum, wherein the microbes in the consortium are arranged and adapted to form a symbiotic relationship to one another in the wastewater upon administering to and growing in the wastewater.

Preferably, the saprophytic yeast has a concentration of 10% to 20% by weight, the photosynthetic microbe has a concentration of 10% to 20% by weight, and the diazotrophic microbe has a concentration of 10% to 20% by weight of the liquid composition. Preferably, the liquid mixture is adjusted to a pH value of 6.5 to 8.5 initially.

The liquid composition of the invention is preferably used for treatment of wastewater from oil production mill, municipal sewage plant, or food-processing plant.

Accordingly, the liquid composition produces an oleaginous biomass from wastewater. The oleaginous biomass is used for production of biocrude oil.

The present invention also describes the use of the liquid composition containing a microbial consortium as described previously for treating wastewater having organic material.

Further, disclosed herein is the use of the liquid composition containing a microbial consortium as set forth in the preceding description for producing an oleaginous biomass for the production of biocrude oil.

### DETAILED DESCRIPTION OF THE INVENTION

One skilled in the art will readily appreciate that the present invention is well adapted to carry out the objects and obtain the ends and advantages mentioned, as well as those inherent therein. The embodiment described herein is not intended as limitations on the scope of the invention.

The present invention discloses a liquid composition containing a microbial consortium for the treatment of wastewater or water containing organic matter, the microbial consortium comprises at least one saprophytic yeast, at least one photosynthetic microbe, and at least one diazotrophic microbe, wherein the microbes in the consortium are arranged and adapted to form a symbiotic relationship in the wastewater upon administering to and growing in the wastewater. The microbial consortium of the invention is genetically unmodified and the combination is more resilient to various external shocks such as temperature, pH changes, nutrient availability and predation due to its biodiversity. It is also intended to integrate along with the wastewater natural flora. Further, it facilitates bioremediation while eliminates the need for additional aeration and chemicals. Besides, the microbial consortium is viable and functionally active in salt water and/or freshwater.

In the preferred embodiment of the invention, the saprophytic yeast in the liquid composition may be selected from the genus Saccharomyces, Cryptococcus, Rhodosporidium, or Yarrowia, or a combination thereof. More preferably, it can be any one of Cryptococcus curvatus (also known as Candida curvata and Trichosporon oleaginosus), Rhodosporidium toruloides, and Yarrowia lipotytica, or a combination thereof. The saprophytic microbe has a concentration ranging from about 10% to about 20% by weight of the liquid composition. Accordingly, saprophytic yeasts produce enzymes responsible for the degradation of organic compounds such as sugar, protein, and lipid components. Further, the preferred saprophytic yeasts compete with methanogens for nutrients, particularly carbon dioxide, thereby suppressing methane emission from wastewater. For example, C. curvatus competes with methanogens for acetic acid or acetate, limiting nutrients available for methanogens. Besides, saprophytic yeasts of the invention are oleaginous strains with high oil production capacity. Therefore, inclusion of these saprophytic microbes in the liquid composition increases the total lipid production with improved bioremediation.

Preferably, the photosynthetic microbe used in the liquid composition is selected from the genus Nannochloropsis, Chlorella, Tetraselmis, Scenedesmus, Rhodopseudomonas, and Rhodobacter. More preferably, any one of Nannochloropsis oculata, Chlorella vulgaris, Tetraselmis suecica, Scenedesmus obliquus, Rhodopseudomonas capsulate, and Rhodobacter sphaeroides, or a combination thereof may be included in the liquid composition. Photosynthetic microbe has a concentration of about 10% to about 20% by weight of liquid composition. Photosynthetic microbes in accordance with the invention consume organic matters in wastewater and thus removing excess nutrients in wastewater meanwhile providing oxygen for aerobic saprophytes. Consequently, the effect of anoxic condition in aerobic wastewater treatment system is minimized. More particularly, photosynthetic microbes of the invention can be microalgae and/or purple non-sulfur bacteria. Microalgae species contemplated within the invention have high oil content, making them suitable candidates for biofuel production. Photosynthetic activity of microalgae consumes carbon dioxide and yields oxygen, thereby adding to the amount of dissolved oxygen in the wastewater. Also, microalgae of the invention are capable of removing heavy metals from wastewater owing to their capability to sorb heavy metals. Microalgae in the liquid composition may or may not be accompanied by purple non-sulfur bacteria (PNSB). PNSB can grow photoautotrophically by consuming carbon dioxide for growth. By competing with methanogens for various forms of carbon sources such as carbon dioxide and acetate, PNSB limit methane production by methanogens, and the fact that they consume carbon dioxide further reduces greenhouse gases in the environment. In addition, PNSB neutralize various toxic and odorous organic compounds in wastewater. Their ability to fix nitrogen even under anoxic condition also increases the viability of the polymicrobial culture. Interestingly, PNSB also aid in denitrification, further cleaning up the wastewater of excess contaminants.

It is preferable to include diazotrophic microbe selected from either one or both of the genus Azotobacter and Azospirillum in the liquid composition. More preferably, Azotobacter chroococcum and Azospirillum brasilense are used. Diazotrophic microbe has a concentration of about 10% to about 20% by weight of liquid composition. Diazotrophic microbes are capable of degrading a wide variety of organic compounds such as polyphenols and phytotoxins in wastewater. They also fix atmospheric nitrogen into more usable form, such as ammonia which then is oxidized into nitrates, and nitrites, eventually increasing the level of dissolved nitrogen in the wastewater. Dissolved nitrogen supports the growth of other microorganisms such as microalgae and saprophytic yeasts especially in the oil mill wastewater where nitrogen is a growth-limiting factor due to scarcity. Besides, oil mill wastewater often contains antimicrobial polyphenols that suppresses microbial activitity, and yet diazotrophic microbe such as Azospirillum has been shown to degrade these antimicrobial polyphenols. Saprophytic yeasts and photosynthetic microbes are thus able to grow in wastewater containing antimicrobial polyphenols. Hence, the presence of diazotrophic microbes in the wastewater enhances oleaginous biomass production and so the total lipid production.

According to the preferred embodiment of the present invention, the liquid composition contains a polyculture of microbes which work symbiotically as a microecosystem. The three categories of microbes disclosed herein create a self-supporting ecosystem wherein microbes of one microbial category support the growth of another microbial category. The liquid composition inoculated into the wastewater would gradually turn the environment into an environment more favorable for the growth of other symbiotic microbes indigenous to the system. Specifically, presence of microbes of one category supplies growth nutrients essential for microbes in other microbial categories that are originally absent in the wastewater. For example, when the wastewater contains abundant organic carbon sources but lacks of or has negligible amount of dissolved nitrogen, growth of diazotrophic microbes is favorable but growth of photosynthetic microbes and saprophytic yeasts is not likely initially. Diazotrophic microbes break down and utilize carbon sources in the wastewater and fix atmospheric nitrogen into more usable forms such as ammonia, which also gets oxidized into nitrates and nitrites that are key growth factors for non-azotrophs. With ample amount of nitrogen fixed, growth of photosynthetic microbes and saprophytic yeasts will follow. Growth of photosynthetic microbes provides oxygen for growth of diazotrophs and saprophytic yeast, meanwhile saprophytic yeasts and diazotrophs provide carbon dioxide in return for photosynthetic microbes. The presence of carbon dioxide also helps to maintain the pH of the wastewater. The polyculture also helps to chelate minerals and improving the bioavailability of the minerals to other microbes in the system. The system eventually becomes more suitable for growth for all microbes thereby increasing the rate of consumption of wastewater contaminants.

The liquid composition containing a microbial consortium disclosed herein is effective in treating wastewater from places including, without limitation, oil production mill, municipal sewage plant, and food-processing plant. The wastewater to be treated comprises mainly of organic matters, which serve as growth substrates for the microbial consortium. Due to the diverse water-types in wastewater treatment, such as high-carbon but low-nitrogen wastewater from vinegar or oil production plants, or high-nitrogen but low-carbon wastewater from meat-processing plants, a polymicrobial culture possessing multiple metabolic modes as disclosed herein would be necessary to effectively treat these various water-type. The diverse metabolic modes and the symbiotic nature of these microorganisms create a microecosystem of its own. This symbiotic nature allows the polymicrobial culture to more effectively consume wastewater nutrients while generating very minimal to negligible wastes into the atmosphere.

The liquid composition in accordance with the invention is prepared by culturing the saprophytic yeast, the photosynthetic microbe, and the diazotrophic microbe individually in specific media until the microbes reach a density of 104 to 107 microbial cells per milliliter of culture. The monocultures are then combined according to a specific ratio in a nutrient broth consisting of minimal nitrogen and carbon sources supplemented with minerals and salt. Preferably, when more than one strain from a microbial category (saprophytic, photosynthetic, or diazotrophic microbe) is included in the liquid composition, equal portion of culture of each strain is used. Despite more than one strain from a microbial category is used, the actual ratio of each microbial category in the liquid composition readjusts itself. This is the same with the pH, which would be adjusted to 7 initially.

The liquid composition containing a microbial consortium disclosed herein is able to produce an oleaginous polymicrobial biomass with lipid content as high as 20% to 55% by weight without the need to genetically modify the microbes. Preferably, the oleaginous biomass is subjected to hydrothermal liquefaction (HTL) process to produce biocrude oil which composition most closely resembles the composition of natural crude oil. Therefore, biocrude oil can be processed with existing infrastructure making this technology economically practical. .

The polymicrobial biomass exploits the benefits of HTL technology, which requires no painstaking harvesting and drying of the oleaginous biomass, not limited by the biodiversity of the biomass and no physical or chemical pretreatment of the biomass before the HTL process (howbeit the presence of a catalyst would increase the yield of biocrude oil). The biomass is kept at 15% to 30% by weight with water as the solvent and is subjected to a temperature ranging between 250 ° C. and 370° C. and a pressure ranging between 10 to 25 MPa. At this temperature and pressure, highly reactive subcritical water promotes the breaking down of the reformation of chemical building blocks for the oleaginous biomass into a biocrude oil.

The invention also describes the use of the liquid composition containing a microbial consortium as set forth in preceding description for treating wastewater having organic waste. The wastewater is inoculated with the liquid composition of the invention. No pH adjustment is necessary as the microbial consortium disclosed herein would by itself adjust the pH to a range of 6.5 to 8.5 suitable for its own growth. Multiple inoculations may be required to facilitate establishment of the consortium in the new wastewater surrounding as they will slowly integrate or compete with the existing flora. The strains in accordance with the invention are able to grow as a free culture. Preferably, about 1 to 4 L of liquid composition is required for treating about 1000 L of wastewater. The wastewater treatment is implemented on a continuous basis unless it is a fully established system. Pursuant to the preferred embodiment of the invention, the liquid composition disclosed herein remove at least 90%, preferably 99%, of organic materials from the wastewater. Meanwhile, oleaginous biomass is produced from wastewater that can be processed into biocrude oil as mentioned in the preceding description.

1. A liquid composition containing a microbial consortium for treatment of wastewater or water containing organic matter, the microbial consortium comprising:
at least one saprophytic yeast selected from genus Saccharomyces, Cryptococcus, Rhodosporidium, and Yarrowia;
at least one photosynthetic microbe selected from genus Nannochloropsis, Chlorella, Tetraselmis, Scenedesmus, Rhodopseudomonas, and Rhodobacter; and
at least one diazotrophic microbe selected from genus Azotobacter and Azospirillum, wherein the microbes in the consortium are arranged and adapted to form a symbiotic relationship to one another in the wastewater.

2. The liquid composition according to claim 1, wherein the saprophytic yeast has a concentration of 10% to 20% by weight of the liquid composition.

3. The liquid composition according to claim 1, wherein the diazotrophic microbe has a concentration of 10% to 20% by weight of the liquid composition.

4. The liquid composition according to claim 1, wherein the photosynthetic microbe has a concentration of 10% to 20% by weight of the liquid composition.

5. The liquid composition according to claim 1, wherein the wastewater is derived from oil production mill, municipal sewage plant, or food-processing plant.

6. The liquid composition according to claim 1, wherein the liquid composition has a pH value ranging from 6.5 to 8.5.

7. The liquid composition according to claim 1, wherein the consortium produces an oleaginous biomass.

8. The liquid composition according to claim 7, wherein the oleaginous biomass is used for production of biocrude oil.

9. Use of liquid composition containing a microbial consortium as set forth in claim 1 for treating wastewater or water having organic matter.

10. Use of liquid composition containing a microbial consortium as set forth in claim 1 for producing an oleaginous biomass for the production of biocrude oil.
